(19) United States
(12) Patent Application Publication
(10) Pub. No.: US 2008/0306388 A1
(43) Pub. Date: Dec. 11, 2008

(54) Non-Strap Treatment Applicator

(75) Inventors: Kevin Jay Tanis, Collierville, TN (US); Debra Ann Arrington, Cordova, TN (US)

Correspondence Address: JOEL R. PETROW;SMITH & NEPHEW, INC., 1450 BROOKS ROAD, MEMPHIS, TN, 38116, US

(73) Assignee: Smith & Nephew, Inc., Memphis, TN (US)

(21) Appl. No.: 12/159,994

(22) PCT Filed: Jan. 8, 2007
(86) PCT No.: PCT/US07/00178

§ 371 Date: Jul. 3, 2008

Related U.S. Application Data
(60) Provisional application No. 60/757,144, filed on Jan. 6, 2006.

(51) Int. Cl. A61B 8/14

(52) U.S. Cl. 600/459

(57) ABSTRACT

An ultrasound transducer assembly including a ultrasound transducer and a transducer holder. The transducer holder may have a central body and a plurality of arms, wherein the lower surfaces of the plurality of arms comprises an adhesive to adhere the ultrasound transducer assembly proximate to a treatment site.

### CROSS-REFERENCE TO RELATED APPLICATIONS

This document claims the benefit of U.S. Provisional Patent Application Ser. No. 60/757,144, entitled “Non-Strap Treatment Applicator” and filed Jan. 6, 2006, the entire contents of which are hereby incorporated by this reference.

### RELATED FIELDS

Embodiments of the present invention may relate to a non-strap treatment applicator for use with ultrasound or other technologies.

### BACKGROUND

Ultrasound has been used as a therapeutic, surgical, and diagnostic tool to treat patients for a wide variety of conditions. For instance, ultrasound treatments have been used as a non-invasive therapy for pathological conditions, such as inflammation of soft tissue.

Some ultrasound procedures utilize an ultrasound transducer positioned proximate a treatment site on the patient. Often, it will be desirable to maintain the position of the transducer with respect to the treatment site for extended periods of time.

To keep the transducer close to the treatment site, oftentimes surgical tape and/or straps are used. However, the use of straps and/or surgical tape is not always effective because desired treatment sites on the body may not be perfectly flat or symmetrical, and therefore, it can be difficult to use only straps and/or surgical tape to maintain the position of the transducer close to the treatment site. In addition, straps and bandages can be difficult to apply using a single hand, which may be particularly problematic for individuals with upper body injuries. Also problematic, the use of surgical tape on highly perspiring treatment areas is cumbersome and ineffective.

### SUMMARY OF THE INVENTION

Embodiments of the present invention may provide improved devices and methods for positioning an ultrasound transducer on or proximate a treatment site. For instance, some embodiments of the present invention may include a transducer and applicator assembly being particularly suited for mounting to difficult to strap treatment sites, such as feet, knees, elbows, shoulders, collarbones, and highly perspiring areas.

Additionally, some embodiments of the present invention may provide a transducer and applicator assembly that enables single handed, fast and effective application to a desired treatment site including conformability on uneven surfaces.

According to an aspect of the present invention there may be provided an ultrasound transducer assembly, comprising an ultrasound transducer and a transducer holder for at least temporarily positioning at least portions of the ultrasound transducer on or proximate to a treatment site. The transducer holder comprising a central body, the central body comprising a cavity for receiving and holding at least portions of the ultrasound transducer. The transducer holder characterized in that the holder further comprises a plurality of arms extending away from the central body, wherein at least one arm is at least partially independently positionable with respect to at least one other arm.

According to some embodiments of the present invention, the lower surfaces of the arms comprise an adhesive, wherein said adhesive at least temporarily adheres the arms to areas comprising or proximate the treatment site.

According to some embodiments of the present invention, the adhesive of the assembly may comprise a non-permanent, reusable adhesive.

According to some embodiments of the present invention, the adhesive of the assembly may comprise a hydro-gel adhesive.

According to some embodiments of the present invention, the holder may comprise at least one removable layer at least partially covering the lower surfaces of the arms, wherein a lower surface of the at least one removable layer comprises a second layer of adhesive.

According to some embodiments of the present invention, the at least one removable layer of the assembly may comprise a plurality of removable layers.

According to some embodiments of the present invention, at least one of the arms of the assembly may be flexible.

According to some embodiments of the present invention, all of the arms of the assembly may be flexible.

According to some embodiments of the present invention, the cavity of the assembly may comprise an aperture extending through the central body.

According to some embodiments of the present invention, the ultrasound transducer of the assembly may be adhered to the transducer holder.

According to some embodiments of the present invention, the arms of the assembly may extend at least somewhat symmetrically from the aperture when the holder is positioned on a substantially flat surface.

According to some embodiments of the present invention, the holder comprises an outer circumference, wherein the outer circumference may be a daisy shape, star shape, sun shape, cross shape, or clover shape.

“Embodiment” as used herein can be considered to mean an aspect or object of the invention, and vice versa.

### BRIEF DESCRIPTION OF THE DRAWINGS

FIG. 1 schematically shows an ultrasound transducer assembly in accordance with a first embodiment of the present invention.

FIG. 2 schematically shows a transducer holder of the ultrasound transducer assembly shown in FIG. 1.

FIG. 3 schematically shows a transducer holder in accordance with another embodiment of the present invention.

FIG. 4 schematically shows a transducer holder in accordance with another embodiment of the present invention.

FIG. 5 schematically shows a cross-sectional view of the transducer holder shown in FIG. 2.

FIG. 6 schematically shows a transducer holder in accordance with another embodiment of the present invention.

FIG. 7 schematically shows a transducer holder in accordance with another embodiment of the present invention.

FIG. 8 schematically shows a bottom-view of the transducer holder shown in FIG. 2.

FIG. 9 schematically shows a transducer holder in accordance with another embodiment of the present invention.

FIG. 10 schematically shows a transducer holder in accordance with another embodiment of the present invention.

### DETAILED DESCRIPTION OF THE DRAWINGS

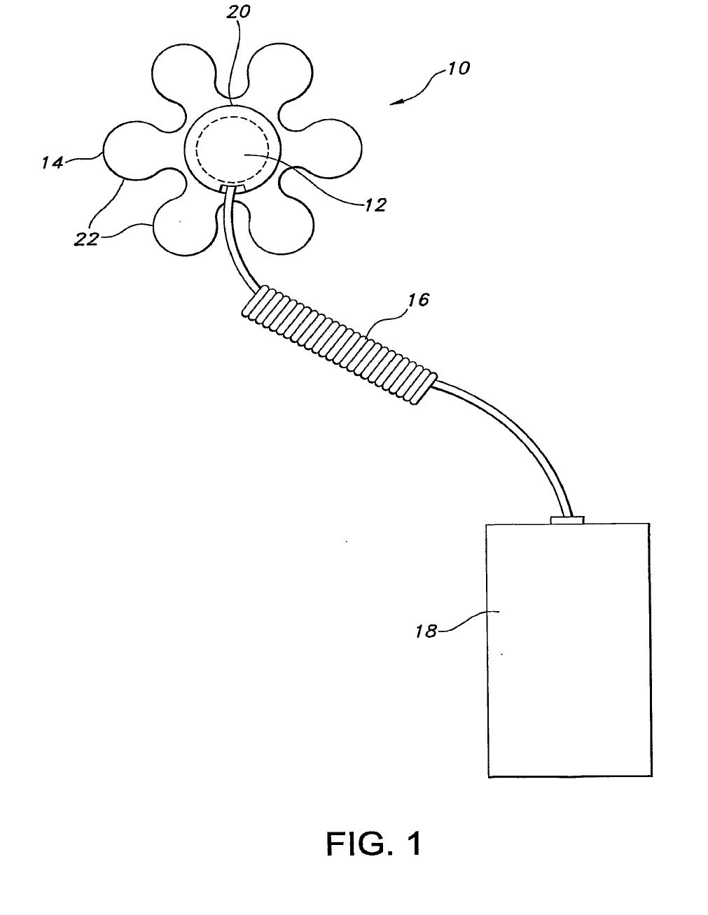

FIG. 1 shows one of the embodiments of the present invention. FIG. 1 shows an ultrasound transducer assembly 10, which includes an ultrasound transducer 12 and a transducer holder 14. The transducer holder 14 may be used to position the ultrasound transducer 12 on or proximate to a treatment site. FIG. 1 shows the ultrasound transducer assembly 10 in use, wherein the ultrasound transducer 12 is connected to a portable, ergonomically constructed main operating unit (MOU) 18 by a cable 16. To permit patient mobility during treatment, the MOU 18 has an internal power source. The MOU 18 has a user interface and provides ultrasound control signals that drive the ultrasonic transducer 12 to produce ultrasound output. Cable 16 is preferably a multi-conductor cable capable of transmitting relatively low frequency or optical signals, as well as digital signals. Cable 16 may include coaxial cable or other type of suitable shielded cable. Alternatively, cable 16 may include fiber optic cable for transmitting optical signals.

The transducer holder 14 shown in FIG. 1 includes a central body 20 and a plurality of arms 22 extending away from the central body 20. The central body 20 may be made from a rigid material. In some embodiments, the central body 20 is made from an ABS plastic. In some other embodiments, the central body 20 may be made from other rigid materials such as, thick high impact plastic (e.g., HDPE), heavy cuir boulli, layers of heavy leather, heavy leather backed by thick closed cell foam, or any other material rigid enough to not impinge on the insertion and removal of the ultrasound transducer 12. In other embodiments, central body 20 does not necessarily have to be rigid and may be flexible.

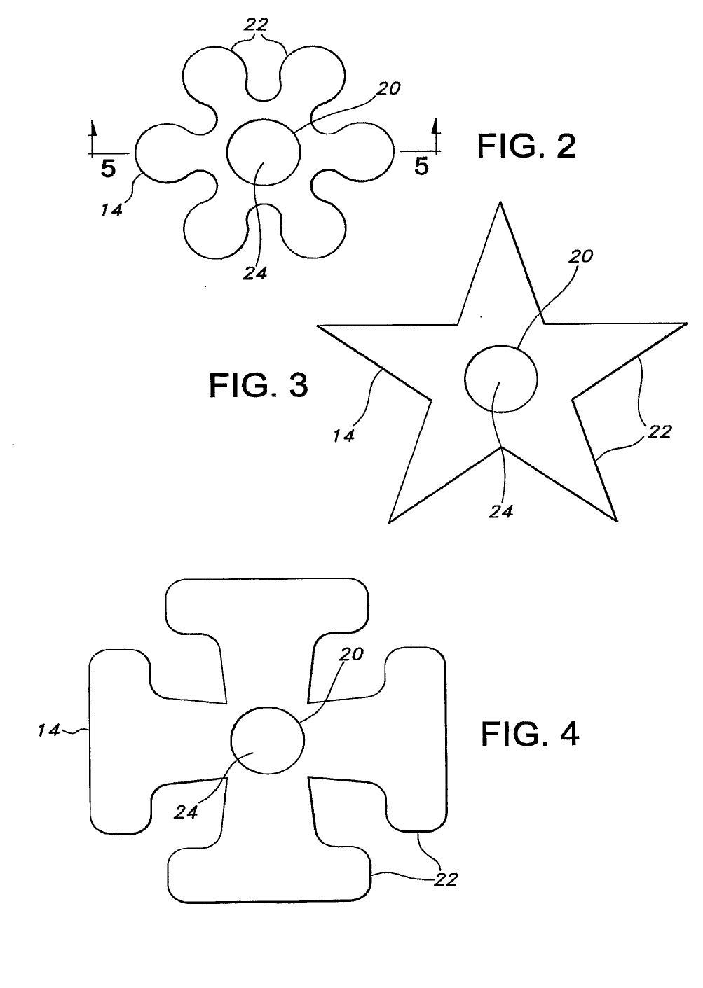

The central body 20 may include a cavity 24 that may be shaped to secure the ultrasound transducer 12 with respect to the arms 22. FIGS. 2, 3, 4, 6 and 7, show that the cavity 24 may be an aperture that extends through the central body 20. Alternatively, the cavity 24 does not have to be an aperture.

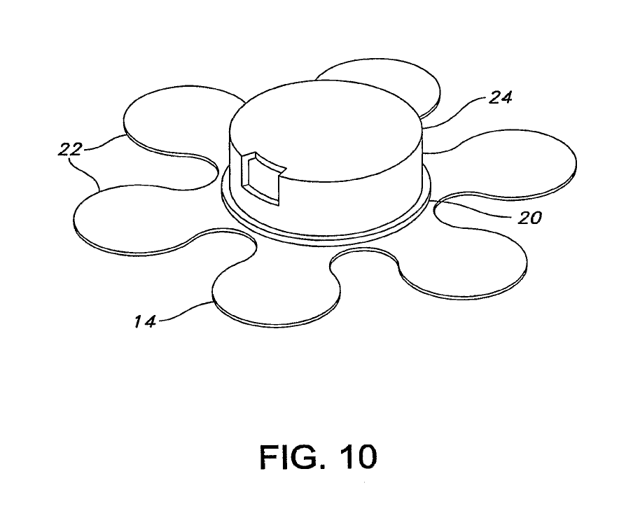

In alternative embodiments, the cavity 24 is molded into a shape that is complimentary to the ultrasound transducer 12 it is intended to secure. As shown in FIG. 10, the cavity 24 may be a cap or a housing, in which to snap the ultrasound transducer 12. In some other embodiments, the ultrasound transducer 12 may be snapped into a separate housing and then attached to the cavity 24.

In other alternative embodiments, the cavity 24 is not molded, but rather, is made from any material having a stretchable characteristic and therefore, inherently will conform generally to the shape of the ultrasound transducer 12. In still other embodiments, cavity 24 may be any other structure or mechanism sufficient to associate the ultrasound transducer 12 with the central body 20.

FIGS. 2, 3, 4, 6 and 7 show that the transducer holder 14 may include a plurality of arms 22 surrounding and extending away from the central body 20. The arms 22 can have any dimensions and the dimensions may vary depending on the desired treatment site that the ultrasound transducer assembly 10 is designed to adhere to. In some embodiments, the arms 22 may be flexible and may be made from fabric, polymer, plastic, or any pliable material that adhesive can be applied to. As shown in FIGS. 2-4 and 6-7, the arms 22 may extend at least somewhat symmetrically from the cavity 24 when the transducer holder 14 is positioned on a flat surface. In other embodiments, arms 22 may extend asymmetrically.

As also shown in FIGS. 2, 3, 4, 6 and 7, the plurality of arms 22 may have various shapes, wherein the outer circumference of the transducer holder 14 could be in a daisy shape, star shape, cross shape, sun shape, clover shape or other shapes. The shapes of the plurality of arms 22 may allow for a maximum adhesive surface to maintain the placement of the transducer assembly 10 throughout the ultrasound treatment period.

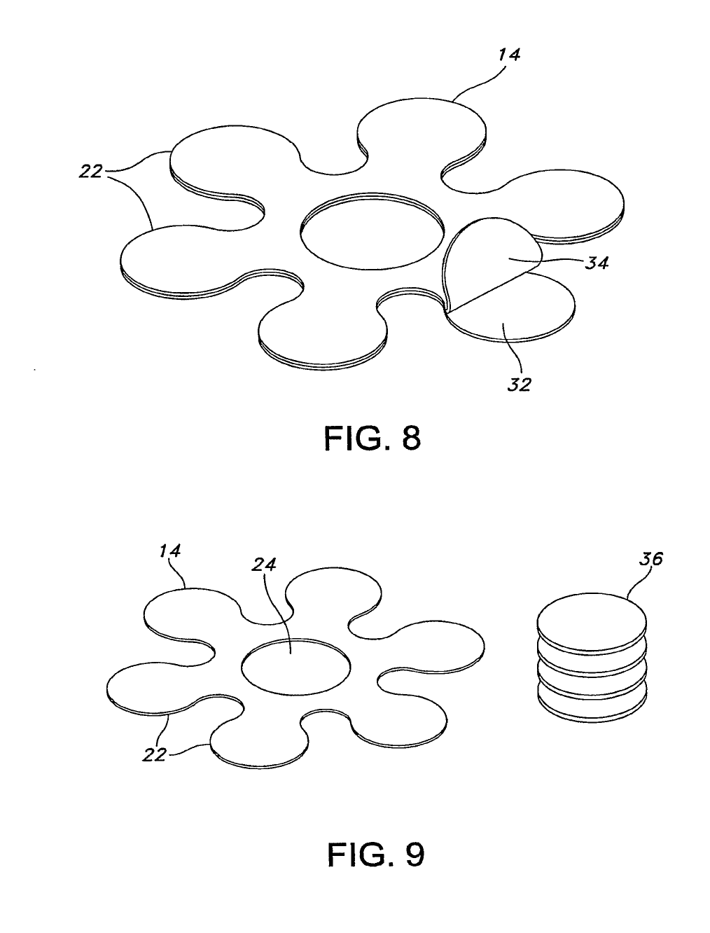

FIG. 8 schematically shows another embodiment of the present invention. FIG. 8 shows a bottom-view of the transducer holder 14, wherein the lower surfaces of the arms 22 may include an adhesive 32, such that the adhesive 32 is on the skin attachment side of the transducer holder 14. The adhesive 32 may be provided across the entire lower surfaces of the arms 22, around the periphery of the lowers surfaces of the arms 22, or in any manner to allow sufficient adhesion on the lower surfaces of the arms 22 in order to hold the transducer assembly 10 in place. As shown in FIG. 8, the adhesive 32 may be pre-applied with a protective peel-off backing 34. In other embodiments, the adhesive 32 may be supplied to the patient as a separate item to apply to the lower surfaces of the arms 22.

Embodiments of the present invention are not limited to particular types of adhesive. In some embodiments, the adhesive 32 may comprise a non-permanent, reusable adhesive. In some embodiments, the adhesive 32 may be a hydro-gel adhesive. In some other embodiments, the adhesive 32 may be a synthetic rubber adhesive, double coated polyethylene tape, polyester fiber filled transfer adhesive, or PET tape. In still other embodiments, the adhesive 32 may be varied depending on the type of treatment site the ultrasound transducer assembly 10 will attach to.

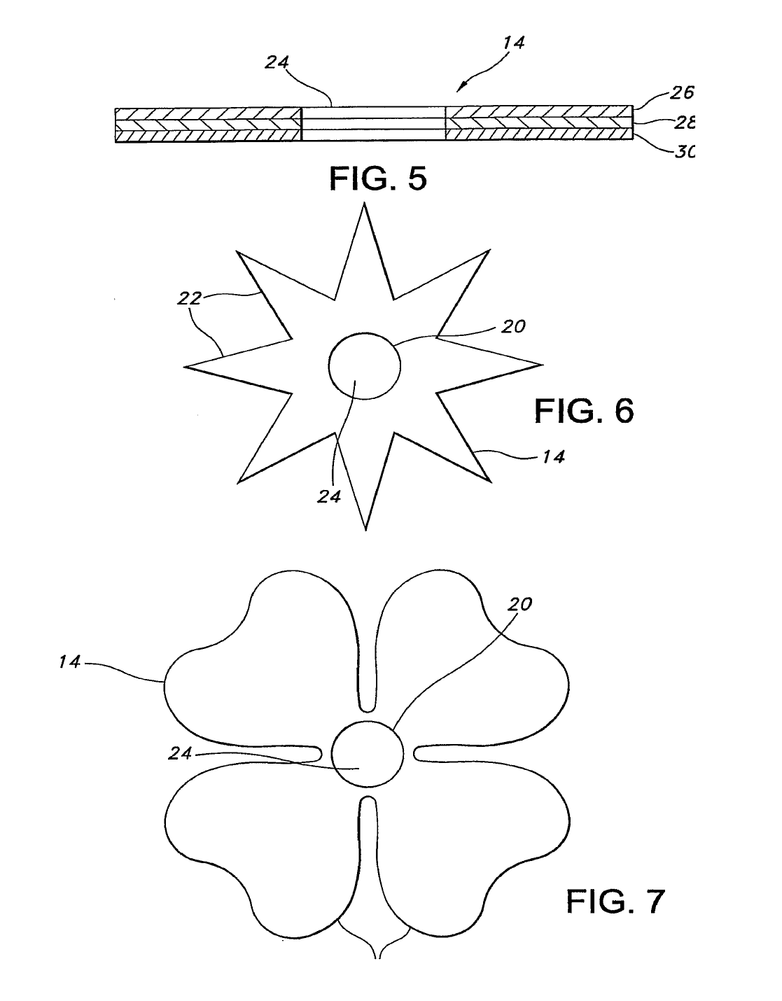

FIG. 5 schematically shows a cross-sectional view of the transducer holder 14, wherein the transducer holder 14 includes removable layers 26, 28, and 30 stacked one on top of another. Each removable layer 26, 28, and 30 may at least partially cover the lower surfaces of the arms 22, such that the lower surfaces of the removable layers 26, 28, and 30 comprise a second layer of adhesive 32. Thus when one removable layer 30 is torn off and disposed of, a fresh removable layer 28 remains for the next application to the desired treatment site, and so on.

In some embodiments, the transducer holder 14 may be manufactured by bonding together the material and adhesive 32, with the adhesive 32 having a protective peel-off backing 34 on it. Then the transducer holder 14 is die cut or stamped out. Other methods of manufacture are also possible.

Transducer holder 14 may be used to position a transducer 12 on or proximate to a treatment site. In use, the ultrasound transducer 12 may be secured in the transducer holder 14 via the central body 20, such as shown in FIG. 1. The ultrasound transducer assembly 10 may then be positioned on or proximate a treatment site (such as a foot, elbow, shoulder, etc.) and secured by the adhesive located on the lower surfaces of the arms 22. The arms 22 may conform to the patient's skin surrounding the treatment site to secure the ultrasound transducer assembly 10. Subsequently, the ultrasound transducer 12 may be powered by the MOU 18 connected by the cable 16 shown in FIG. 1. Thus, the patient can receive ultrasound treatment on the desired treatment site, by use of the ultrasound transducer assembly 10 held securely in place.

In some other embodiments, as shown in FIG. 9, the transducer holder 14 may also include a preformed gel pad 36 located in the cavity 24 for application between the ultrasound transducer 12 and the desired treatment site. The preformed gel pad 36 may be removable from the cavity 24 of the transducer holder 14 to be washed after every treatment. In still other embodiments, the transducer holder 14 may be made from a washable material, so the integral preformed gel pad 36 can be washed after every treatment.

FIG. 5 further illustrates how one embodiment of the present invention may be used for multiple applications. In the example described above, after receiving the desired ultrasound treatment session for the proper amount of time, it may be desired to relocate the ultrasound transducer assembly 10 from one treatment site to another. If so, FIG. 5 shows that the used removable layer 30 may be torn off, and then a fresh removable layer 28 remains allowing the ultrasound transducer assembly 10 to be placed on yet another desired treatment site and secured in place. The embodiment of the present invention may also be used to apply the ultrasound transducer assembly 10 to the same treatment site multiple times, such as for daily ultrasound treatment applications. In still other embodiments, multiple layers may not be necessary and the adhesive 32 may allow repositioning of the holder 14.

While some of the principles of the invention have been described herein, those of skill in the art will understand that this description is made only by way of example and not as a limitation as to the scope of the invention. Other embodiments are contemplated as within the scope of the present invention in addition to the exemplary embodiments shown and described herein. Modifications and substitutions by one of ordinary skill in the art are considered to be within the scope of the present invention, which is not limited except by claims.

1. An ultrasound transducer assembly, comprising an ultrasound transducer and a transducer holder for a least temporarily positioning at least portions of the ultrasound transducer on or proximate to a treatment site, the transducer holder comprising a central body, the central body comprising a cavity for receiving and holding at least portions of the ultrasound transducer, the transducer holder characterized in that the holder further comprises a plurality of arms extending away from the central body, wherein at least one arm is at least partially independently positionable with respect to at least one other arm.

2. The assembly of claim 1, wherein the lower surfaces of the arms comprise an adhesive, wherein said adhesive at least temporarily adheres the arms to areas comprising or proximate the treatment site.

3. The assembly of claim 2, wherein the adhesive comprises a non-permanent, reusable adhesive.

4. The assembly of claim 3, wherein the adhesive comprises a hydro-gel adhesive.

5. The assembly of claim 2, wherein the holder further comprises at least one removable layer at least partially covering the lower surfaces of the arms; wherein a lower surface of the at least one removable layer comprises a second layer of adhesive.

6. The assembly of claim 5, wherein the at least one removable layer comprises a plurality of removable layers.

7. The assembly of claim 1, wherein at least one of the arms is flexible.

8. The assembly of claim 7, wherein all of the arms are flexible.

9. The assembly of claim 1, wherein the cavity comprises an aperture extending through the central body.

10. The assembly of claim 9, wherein the ultrasound transducer is adhered to the transducer holder.

11. The assembly of claim 9, wherein the arms extend at least somewhat symmetrically from the aperture when the holder is positioned on a flat surface.

12. The assembly of claim 11, wherein the holder further comprises an outer circumference; and wherein the outer circumference is a daisy shape, star shape, sun shape, cross shape, or clover shape.

13. An ultrasound transducer assembly comprising:
an ultrasound transducer; and
a transducer holder for at least temporarily positioning a least portions of the ultrasound transducer on or proximate to a treatment site, the transducer holder comprising:
a central body having a cavity for receiving and holding at least portions of the ultrasound transducer;
a plurality of arms extending away from the central body, wherein at least one arm is at least partially independently positionable with respect to at least one other arm; and
an outer circumference selected from the group consisting of a daisy shape, star shape, sun shape, cross shape, or clover shape.

14. An ultrasound transducer assembly comprising:
an ultrasound transducer; and
a transducer holder for at least temporarily positioning at least portions of the ultrasound transducer on or proximate to a treatment site, the transducer holder comprising:
a central body having a cavity for receiving and holding at least portions of the ultrasound transducer;
a plurality of arms extending away from the central body, wherein at least one arm is at least partially independently positionable with respect to at least one other arm;
each of the plurality of arms having a lower surface, and at least some of the lower surfaces comprise an adhesive, said adhesive at least temporarily adheres the arms to areas comprising or proximate the treatment site.
